(19) United States
(12) Patent Application Publication
(10) Pub. No.: US 2026/0011429 A1
(43) Pub. Date: Jan. 8, 2026

(54) Associating a designated user with a medicament delivery device

(71) Applicant: SHL Medical AG, Zug (CH)

(72) Inventors: Ola Hallström, Nacka Strand (SE); Daniel Carlsson, Nacka Strand (SE); Rasmus Renstad, Nacka Strand (SE)

(21) Appl. No.: 18/992,018

(22) PCT Filed: Jun. 28, 2023
(86) PCT No.: PCT/EP2023/067592

§ 371 Date: Jan. 7, 2025

(30) Foreign Application Priority Data
Jul. 12, 2022 (EP) 22184334.5

(51) Int. Cl. G16H 20/17; A61M 5/31

(52) U.S. Cl. CPC G16H 20/17; A61M 5/31; A61M 2205/3584; A61M 2205/6009

(57) ABSTRACT

The present disclosure provides a method of a receiver of associating a designated user with a medicament delivery device configured to communicate a medicament delivery event wirelessly to the receiver. The method comprises receiving a wireless signal identifying a medicament delivery device communicating a medicament delivery event, acquiring information indicating whether or not at least one neighbouring receiver also has received a wireless signal identifying said medicament delivery device communicating the medicament delivery event, and if not determining that the identified medicament delivery device was used by the designated user for causing the medicament delivery event.

### CROSS-REFERENCE TO RELATED APPLICATIONS

The present application is a U.S. National Phase Application pursuant to 35 U.S.C. § 371 of International Application No. PCT/EP2023/067592 filed Jun. 28, 2023, which claims priority to EP patent application Ser. No. 22/184,334.5 filed Jul. 12, 2022. The entire disclosure contents of these applications are herewith incorporated by reference into the present application.

### TECHNICAL FIELD

The present disclosure relates to methods of a receiver of associating a designated user with a medicament delivery device configured to communicate a medicament delivery event wirelessly to the receiver, and receivers performing the methods.

### BACKGROUND

Medicament delivery devices such as for instance injectors are commonly used or delivering medicament to users suffering for instance from diabetes.

The medicament delivery device may be arranged with a tag for wireless communication of adherence data related to an injection therapy provided by the medicament delivery device, such as for example information specifying time stamp of the medicament delivery event and subsequent disposal indicating the medicament dose having been delivered. identification of dose delivery or cap removal, or other event associated with the device, batch number, type of drug or device, expiry date of medicament, etc. The tag configured to wirelessly communicate the adherence data may be a so-called radio-frequency identification (RFID), a near-field communication (NFC) tag, or even Bluetooth.

When reading the adherence data of the used medicament delivery device with an appropriate wireless receiver in for instance a home of a designated user being associated with the receiver, there may be numerous other injectors capable of wireless communication in the vicinity of the wireless receiver, used for instance by members of the same household or even neighbours.

Thus, even if the wireless receiver receives a unique medicament delivery device identifier along with the adherence data, the receiver cannot ensure that the identified medicament delivery device and the adherence data belongs to the designated user associated with the receiver, and an erroneous registration of a medical event of e.g. a family member or a neighbour may be performed by the receiver.

### SUMMARY

One objective is to solve, or at least mitigate, this problem in the art and to provide a method of a receiver of associating a designated user with a medicament delivery device configured to communicate a medicament delivery event wirelessly to the receiver.

This objective is attained in a first aspect by a method of a receiver of associating a designated user with a medicament delivery device configured to communicate a medicament delivery event wirelessly to the receiver. The method comprises receiving a wireless signal identifying a medicament delivery device communicating a medicament delivery event, acquiring information indicating whether or not at least one neighbouring receiver also has received a wireless signal identifying said medicament delivery device communicating the medicament delivery event, and if not determining that the identified medicament delivery device was used by the designated user for causing the medicament delivery event.

This objective is attained in a second aspect by a receiver configured to associate a designated user with a medicament delivery device configured to communicate a medicament delivery event wirelessly to the receiver. The receiver comprises a processing unit and a memory, said memory containing instructions executable by said processing unit, whereby the receiver is operative to receive a wireless signal identifying a medicament delivery device communicating a medicament delivery event, acquire information indicating whether or not at least one neighbouring receiver also has received a wireless signal identifying said medicament delivery device communicating the medicament delivery event, and if not to determine that the identified medicament delivery device was used by the designated user for causing the medicament delivery event.

Hence, after having received a signal identifying a medicament delivery device and thus that a medicament delivery event has occurred, the receiver communicates with one or more neighbouring receivers, either directly or via a network communication capability (such as a back-end cloud server in communication with the receivers), in order to acquire information indicating whether or not the medicament delivery event of the medicament delivery device also was registered with the neighbouring receiver.

If not, the receiver will advantageously conclude that the designated user indeed is the user that triggered the medicament delivery event using the medicament delivery device and that any adherence data just received from the medicament delivery device hence belongs to the designated user.

In an embodiment, if the acquired information indicates that a wireless signal identifying said medicament delivery device has been received at said at least one neighbouring received, a confirmation is requested from the designated user indicating whether or not the user triggered the medicament delivery event, wherein it is determined that the identified medicament delivery device was used by the designated user for causing the medicament delivery event if a confirmation is received from the user in response to the request.

In an embodiment, the requesting and receiving of the confirmation is effected over a user interface of the receiver.

In an embodiment, the received confirmation further comprises an identifier of the medicament delivery device for which the designated user triggered the medicament delivery event.

In an embodiment, if wireless signals are received identifying a plurality of medicament delivery devices communicating a medicament delivery event, a recommendation is provided to the designated user to change medicament delivery schedule.

In an embodiment, if the acquired information indicates that a wireless signal identifying said medicament delivery device has been received at said at least one neighbouring receiver the neighbouring device is informed of the received confirmation.

The objective is attained in a third aspect by a method of a receiver of associating a designated user with a medicament delivery device configured to communicate a medicament delivery event wirelessly to the receiver. The method comprises receiving a wireless signal identifying a medicament delivery device communicating a medicament delivery event, determining whether or not the identification of the medicament delivery device complies with a predetermined identification number pattern, and if so determining that the identified medicament delivery de

[symbol]

The objective is attained in a fourth aspect by a receiver configured to associate a designated user with a medicament delivery device configured to communicate a medicament delivery event wirelessly to the receiver. The receiver comprises a processing unit and a memory, said memory containing instructions executable by said processing unit, whereby the receiver is operative to receive a wireless signal identifying a medicament delivery device communicating a medicament delivery event, determine whether or not the identification of the medicament delivery device complies with a predetermined identification number pattern, and if so to determine that the identified medicament delivery device was used by the designated user for causing the medicament delivery event.

Advantageously, the receiver will conclude that an identified medicament delivery device was used by the designated user for causing the medicament delivery event only if the device identification information complies with a predetermined identification number pattern.

In an embodiment, the identification of the medicament delivery device is determined to comply with a predetermined identification number pattern if the identification of the medicament delivery device follows a predetermined identification number series.

In an embodiment, the identification of the medicament delivery device is determined to comply with a predetermined identification number pattern if said identification has been pre-registered with the receiver.

In a fifth aspect, a computer program comprising computer-executable instructions is provided for causing a receiver of the second and fourth aspect to perform steps recited in the method according to the first aspect and third aspect, respectively, when the computer-executable instructions are executed on a processing unit included in the receiver.

In a sixth aspect, a computer program product is provided comprising a computer readable medium, the computer readable medium having the computer program according to the sixth aspect embodied thereon.

Generally, all terms used in the claims are to be interpreted according to their ordinary meaning in the technical field, unless explicitly defined otherwise herein. All references to “a/an/the element, apparatus, component, means, step, etc.” are to be interpreted openly as referring to at least one instance of the element, apparatus, component, means, step, etc., unless explicitly stated otherwise. The steps of any method disclosed herein do not have to be performed in the exact order disclosed, unless explicitly stated.

### BRIEF DESCRIPTION OF THE DRAWINGS

Aspects and embodiments are now described, by way of example, with reference to the accompanying drawings, in which:

FIG. 1 illustrates a prior art scenario;

FIG. 2 illustrates a receiver of an embodiment;

FIG. 3 shows a flowchart illustrating a method of a receiver of associating a designated user with a medicament delivery device configured to communicate a medicament delivery event wirelessly to the receiver according to an embodiment;

FIG. 4 shows a flowchart illustrating a method of a further embodiment;

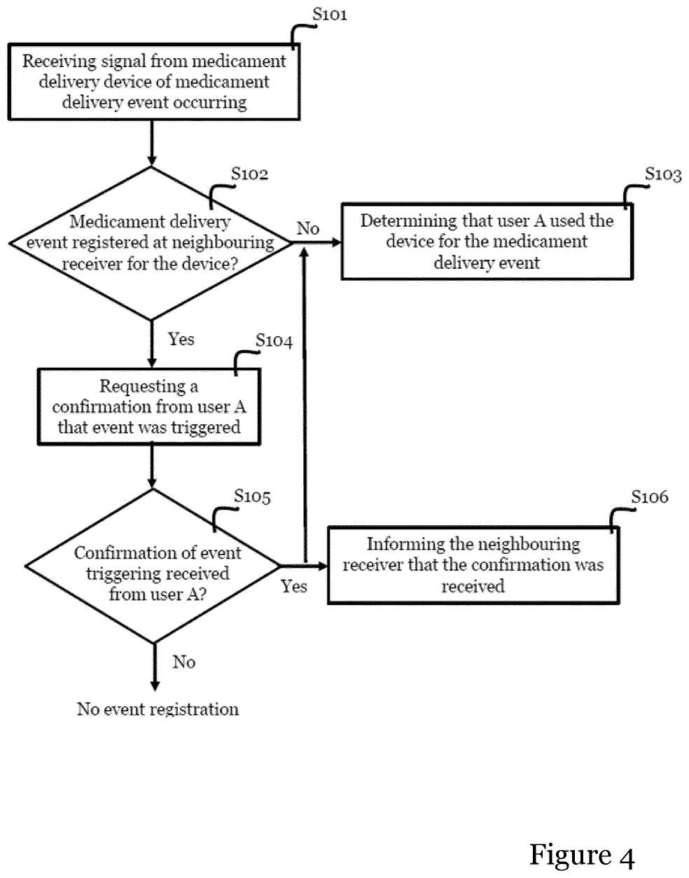

FIG. 5 illustrates a receiver of a further aspect;

FIG. 6 shows a flowchart illustrating a method of yet a further embodiment;

FIG. 7 illustrates a receiver in a further setup; and

FIG. 8 illustrates a receiver of a further embodiment.

### DETAILED DESCRIPTION

The aspects of the present disclosure will now be described more fully hereinafter with reference to the accompanying drawings, in which certain embodiments of the invention are shown.

These aspects may, however, be embodied in many different forms and should not be construed as limiting; rather, these embodiments are provided by way of example so that this disclosure will be thorough and complete, and to fully convey the scope of all aspects of invention to those skilled in the art. Like numbers refer to like elements throughout the description.

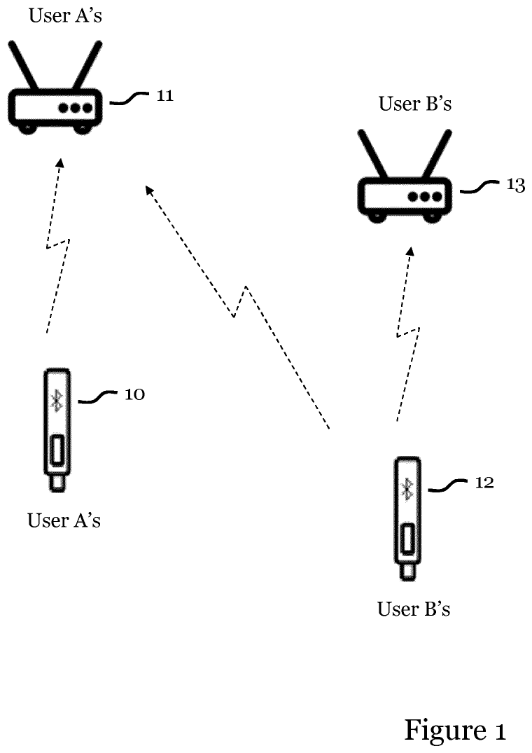

FIG. 1 illustrates a prior art scenario where upon a user utilizing a first medicament device 10, in the following exemplified in the form of an injector, for delivering medicament, the first injector 10 wirelessly transmits a signal to a first wireless receiver 11 that a medicament delivery event has occurred along with appropriate data (commonly referred to as adherence data) and an identifier for the first injector 10.

Upon receiving the signal, the first receiver 11 concludes from the identifier that the signal originates from the first device 10 and will further assume that the medicament delivery event was triggered by a designated user associated with the first receiver 11, in the following referred to as user A.

As mentioned, the wireless communication may be established using any appropriate wireless channel, such as e.g. RFID, NFC, Bluetooth, long range (LoRa) technology or ultra-wideband (UWB) radio.

However, assuming that a second injector 12 communicating with a second wireless receiver 13 is in a vicinity of the first receiver 11. The second injector 12 and second receiver 13 may be used by a household member or a neighbour depending on wireless signal range, referred to as user B being associated with the second receiver 13.

Hence, it may occur that the first receiver 11 is within the range of the second injector 12, and the first receiver 11 receiving the identifier and adherence data of the second injector 12 erroneously assumes that the medicament delivery event was triggered by user A.

As a consequence, in addition to incorrectly registering the medicament delivery event of the second injector 12 as being triggered by user A, the first receiver will further receive incorrect adherence data which really belongs to user B. As is understood, the second receiver 13 may erroneously register a medicament delivery even of user A by receiving signals from the first injector 10.

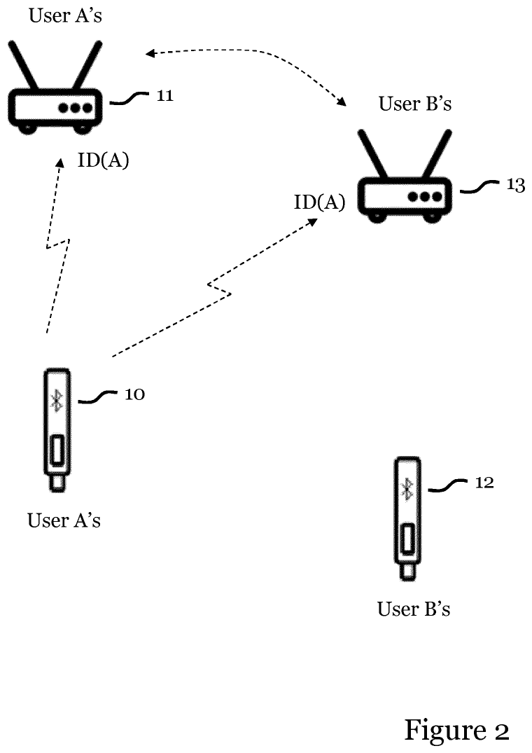

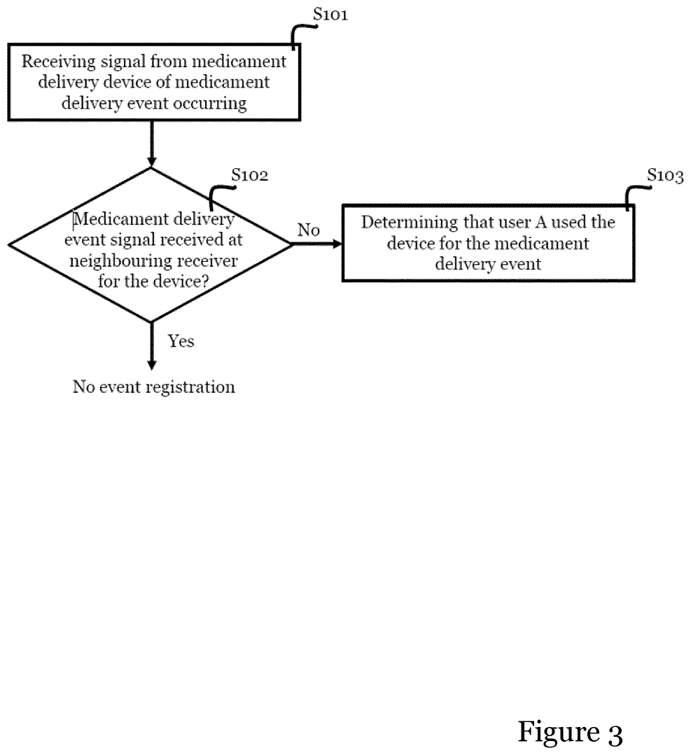

To resolve this issue, reference is made to FIG. 2 illustrating an embodiment. Reference is further made to FIG. 3 showing a flowchart illustrating a method of a receiver (i.e. first receiver 11) of associating a designated user (i.e. user A) with a medicament delivery device (i.e. first injector 10) configured to communicate a medicament delivery event wirelessly to the receiver.

Hence, in a first step S101, the first wireless receiver 11 receives a signal identifying the first injector 10 and thus that a medicament delivery event has occurred.

Thereafter, the first receiver 11 communicates in step S102 with one or more neighbouring receivers, in this embodiment exemplified by the second receiver 13, either directly as shown in FIG. 2 or via a network communication capability (such as a back-end cloud server in communication with the receivers), in order to acquire information indicating whether or not the medicament delivery event of the first injector 10 also was registered with the second receiver 13.

If not, the first receiver 11 will advantageously conclude in step S103 that user A indeed is the user that triggered the medicament delivery event using the first injector 10 (and that any adherence data just received from the first injector 10 hence belongs to the user A).

In practice, the first receiver 11 will in step S103 register the communicated medicament delivery event with user A.

In contrast, should the medicament delivery event of the first injector 10 also be registered at the second receiver 13 as determined in step S102, the first receiver 11 will request a confirmation in step S104 from user A that user A indeed just triggered a medicament delivery event. If user A provides such a conformation, the first receiver 11 concludes that the user that triggered the medicament delivery event using the first injector 10.

If user A does not provide such confirmation in step S104, the first receiver 11 will determine that the signal just received from the first injector 10 was not caused by user A triggering a medicament delivery event at the first injector 10 and registration will be made at the first receiver 11.

In an embodiment, the first receiver 11 is equipped with a user interface via which the first receiver 11 may request a confirmation from user A.

For instance, in one implementation, the first receiver 11 may emit a light signal via for instance a light emitting diode (LED), and user A pushes a button on the first receiver 11 as a confirmation that user A just triggered a medicament delivery event.

In a more elaborate implementation, the first receiver 11 may be equipped with a display plus a keypad, or a touch screen, via which the user may be alerted and in response provide a confirmation.

With such more elaborate user interface, user A may also provide the first receiver 11 with the identifier of the first injector 10 (typically indicated on the injector) such that the first receiver 11 is ensured that the first injector 10 indeed was utilized by user A for triggering the medicament delivery event.

Advantageously, by having user A providing a confirmation that the first injector 10 indeed was triggered, any signal received from another device (e.g. the second injector 12, cf. FIG. 1) may be disregarded at the first receiver 11 and an incorrect registration of the medicament delivery event of said another device can be cancelled. Alternatively, a registration is made only if the confirmation is received.

In a further embodiment, the first receiver 11 informs the second receiver 13 of the confirmation received from user A, such that the second receiver 13 may discard any potential registration caused by the received signal identifying the medicament delivery event of the first injector 10.

In an embodiment, if the first receiver 11 receives signals indicating medicament delivery events of the first injector 10 and the second injector 12 occurring at the same time, the first receiver 11 may inform user A of a recommended change in medicament delivery schedule to avoid having to trigger medicament delivery events at a same or similar time as user B. The recommendation of schedule change may be provided to the user over the user interface of the first receiver 11.

For instance, if user A and user B appears to have the same or similar schedule, say Mondays between 12:00 and 13:00, the first receiver 11 may recommend that the user A should change schedule and even give an explicit proposal, such as Tuesdays between 12:00 and 13:00.

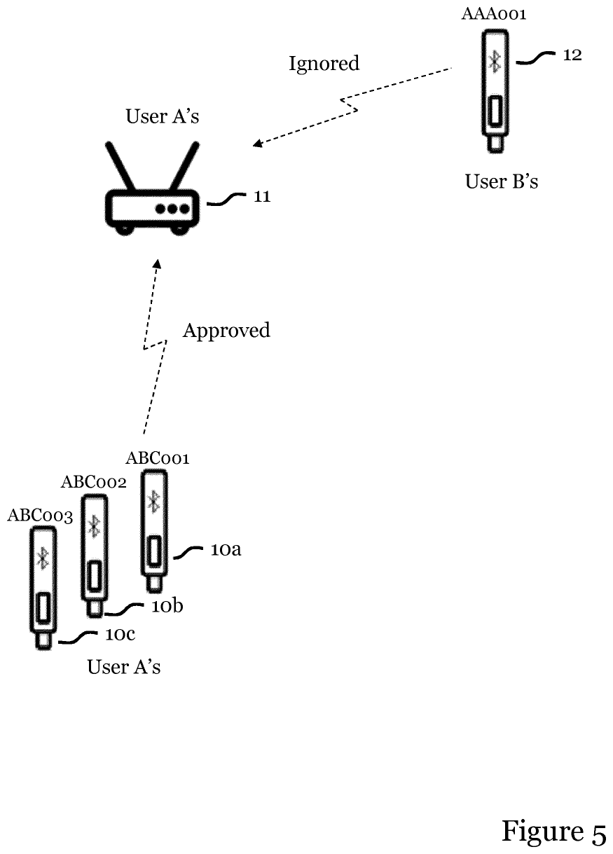

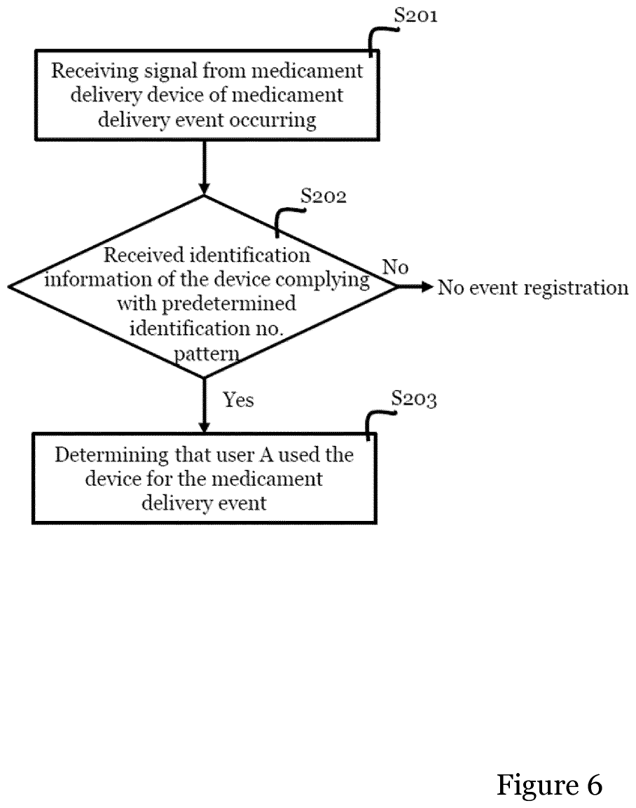

In another aspect, with reference to FIGS. 5 and 6, the first receiver 11 will conclude that an identified medicament delivery device 10a, 10b, 10c was used by the designated user for causing the medicament delivery event only if the device identification information complies with a predetermined identification number pattern.

For instance, as illustrated in FIG. 5, only injectors 10a, 10b, 10c having identification numbers ABC001, ABC002 and ABC003, respectively, will be approved.

Any signalled medicament delivery event from the second injector 12 having identification number AAA001 will thus be ignored, since that particular number does not comply with the predetermined pattern.

Hence, with reference to the flowchart of FIG. 6, in a first step S201 the first wireless receiver 11 receives a signal identifying the first injector 10a and thus that a medicament delivery event has occurred.

Thereafter, the first receiver 11 determines in step S202 whether identification number ABC001 complies with the predetermined pattern, which it in this exemplifying embodiment is assumed to do.

The first receiver 11 will thus advantageously conclude in step S203 that user A indeed is the user that triggered the medicament delivery event using the first injector 10a (and that any adherence data just received from the first injector 10a hence belongs to the user A) and register the communicated medicament delivery event with user A.

In case of the first receiver 11 receiving a medicament delivery event signal from the second injector 12 having identification number AAA001, the event will not be registered since that particular number does not comply with the predetermined pattern.

As is understood, any predetermined identification number pattern with which the first receiver 11 is configured may be envisaged. For instance, it may be assumed that the numbers should be read in rising order starting with ABC001 and ending with ABC003, or in falling order starting with ABC003 and ending with ABC001, and the user may be instructed to use the injectors according to the particular configured pattern.

In another alternative, as long as any number in the series ABC001-ABC003 (typically a packaging comprises far more injectors than three) is presented, the first receiver 11 will approve the number.

To avoid user A and user B having the same identification numbers for the injectors, it is possible to differentiate for instance between weekly batches of injector packagings supplied to a pharmacy. User A picking up the injectors e.g. week 5 would then probably have a different set of identification numbers compared to user B picking up the injectors week 6 and the packagings received by the users could be separated even if multiple packagings are picked up by the same user as long as the two users do not pick up their injectors the same week.

In an embodiment, the identification of the medicament delivery device 10a, 10b, 10c is determined to comply with a predetermined identification number pattern as determined in step S202 if the identification has been pre-registered with the first receiver 11.

In other words, identification numbers ABC001, ABC002 and ABC003 of injectors 10a, 10b and 10c are preregistered with the first receiver 11, and the first receiver 11 will thus approve any of the injectors 10a, 10b and 10c, but not the second injector 12.

The identification numbers may be manually registered by the user with the first receiver 11 using e.g. a keypad of the first receiver 11, or by using a device such as a smart phone. Further, it may be envisaged that the packaging comprising the injectors is equipped with e.g. an RFID tag or QR code from which the first receiver 11 reads all the identification numbers during a preregistration procedure.

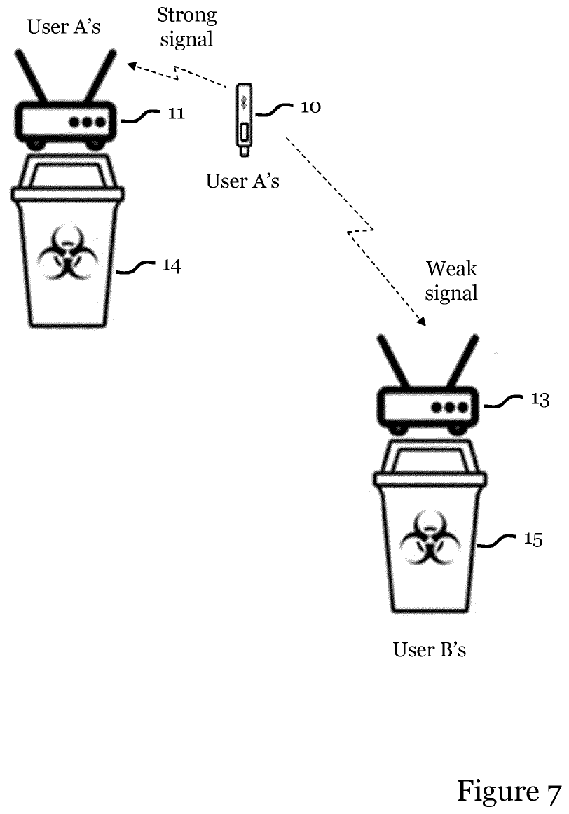

FIG. 7 shows a further setup where the first receiver 11 and the second receiver 13 is arranged on or close to a respective disposal container 14, 15 associated with user A and user B. Thus, if the first injector 10 is disposed in first container 14, the first receiver 11 will experience a strong signal, while second container 15 will experience a weak signal and vice versa. It may be envisaged e.g. that if the signal strength at the first receiver 11 exceeds a threshold value, the first receiver 11 will conclude that user A disposed to first injector 10.

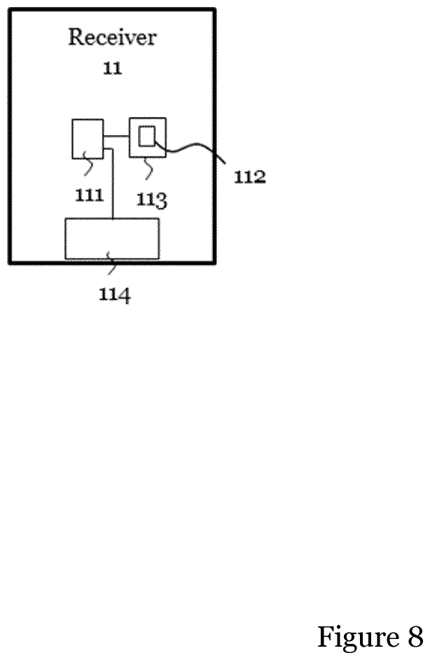

FIG. 8 illustrates a receiver 11 configured to associate a designated user with a medicament delivery device configured to communicate a medicament delivery event wirelessly to the receiver 11 according to an embodiment, where the steps of the method performed by the receiver 11 in practice are performed by a processing unit 111 embodied in the form of one or more microprocessors arranged to execute a computer program 112 downloaded to a storage medium 113 associated with the microprocessor, such as a Random Access Memory (RAM), a Flash memory or a hard disk drive. The processing unit 111 is arranged to cause the receiver 11 to carry out the method according to embodiments when the appropriate computer program 112 comprising computer-executable instructions is downloaded to the storage medium 113 and executed by the processing unit 111. The storage medium 113 may also be a computer program product comprising the computer program 112. Alternatively, the computer program 112 may be transferred to the storage medium 113 by means of a suitable computer program product, such as a Digital Versatile Disc (DVD) or a memory stick. As a further alternative, the computer program 112 may be downloaded to the storage medium 113 over a network. The processing unit 111 may alternatively be embodied in the form of a digital signal processor (DSP), an application specific integrated circuit (ASIC), a field-programmable gate array (FPGA), a complex programmable logic device (CPLD), etc. The receiver 11 further comprises a communication interface 114 (wired and/or wireless) over which the receiver 11 is configured to transmit and receive data using e.g. RFID, NFC, Bluetooth, LoRa technology or UWB radio as previously mentioned.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-la (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

The aspects of the present disclosure have mainly been described above with reference to a few embodiments and examples thereof. However, as is readily appreciated by a person skilled in the art, other embodiments than the ones disclosed above are equally possible within the scope of the invention, as defined by the appended patent claims.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

Some aspects of the invention are outlined in the following clauses.
1. A method of a receiver (11) of associating a designated user with a medicament delivery device configured to communicate a medicament delivery event wirelessly to the receiver (11), comprising:
receiving (S101) a wireless signal identifying a medicament delivery device (10) communicating a medicament delivery event;
acquiring (S102) information indicating whether or not at least one neighbouring receiver (13) also has received a wireless signal identifying said medicament delivery device (10) communicating the medicament delivery event; and if not:
determining (S103) that the identified medicament delivery device (10) was used by the designated user for causing the medicament delivery event.
2. The method of clause 1, wherein if the acquired information indicates that a wireless signal identifying said medicament delivery device (10) has been received at said at least one neighbouring receiver (13), the method further comprises:
requesting (S104) a confirmation from the designated user indicating whether or not the user triggered the medicament delivery event, wherein it is determined (S103) that the identified medicament delivery device (10) was used by the designated user for causing the medicament delivery event if a confirmation is received (S105) from the user in response to the request.
3. The method of clause 2, wherein the requesting (S104) and receiving (S105) of the confirmation is effected over a user interface of the receiver (11).
4. The method of clause 3, the received (S105) confirmation further comprising an identifier of the medicament delivery device (10) for which the designated user triggered the medicament delivery event.
5. The method of clause 3, further comprising, if wireless signals are received identifying a plurality of medicament delivery devices (10, 12) communicating a medicament delivery event:
providing a recommendation to the designated user to change medicament delivery schedule.
6. The method of any one of clauses 2-5, further comprising, if the acquired information indicates that a wireless signal identifying said medicament delivery device (10) has been received at said at least one neighbouring receiver (13):
informing (S106) the neighbouring device (13) of the received confirmation.
7. A method of a receiver (11) of associating a designated user with a medicament delivery device configured to communicate a medicament delivery event wirelessly to the receiver (11), comprising:
receiving (S201) a wireless signal identifying a medicament delivery device (10a, 10b, 10c) communicating a medicament delivery event;
determining (S202) whether or not the identification of the medicament delivery device (10a, 10b, 10c) complies with a predetermined identification number pattern; and if so:
determining (S203) that the identified medicament delivery device (10a, 10b, 10c) was used by the designated user for causing the medicament delivery event.
8. The method of clause 7, the identification of the medicament delivery device (10a, 10b, 10c) being determined to comply with a predetermined identification number pattern if the identification of the medicament delivery device (10a, 10b, 10c) follows a predetermined identification number series.
9. The method of clause 7, the identification of the medicament delivery device (10a, 10b, 10c) being determined to comply with a predetermined identification number pattern if said identification has been pre-registered with the receiver (11).
10. A computer program (112) comprising computer-executable instructions for causing a receiver (11) to perform steps recited in any one of clauses 1-9 when the computer-executable instructions are executed on a processing unit (111) included in the receiver (11).
11. A computer program product comprising a computer readable medium (113), the computer readable medium having the computer program (112) according to clause 10 embodied thereon.
12. A receiver (11) configured to associate a designated user with a medicament delivery device configured to communicate a medicament delivery event wirelessly to the receiver (11), the receiver (11) comprising a processing unit (111) and a memory (113), said memory containing instructions (112) executable by said processing unit (111), whereby the receiver (11) is operative to:
receive a wireless signal identifying a medicament delivery device (10) communicating a medicament delivery event;
acquire information indicating whether or not at least one neighbouring receiver (13) also has received a wireless signal identifying said medicament delivery device (10) communicating the medicament delivery event; and if not to:
determine that the identified medicament delivery device (10) was used by the designated user for causing the medicament delivery event.
13. The receiver (11) of clause 12, further being operative to, if the acquired information indicates that a wireless signal identifying said medicament delivery device (10) has been received at said at least one neighbouring receiver (13):
request a confirmation from the designated user indicating whether or not the user triggered the medicament delivery event, wherein it is determined that the identified medicament delivery device (10) was used by the designated user for causing the medicament delivery event if a confirmation is received from the user in response to the request.
14. The receiver (11) of clause 13, further being operative to request and receive the confirmation over a user interface of the receiver (11).
15. The receiver (11) of clause 14, the received confirmation further being configured to comprise an identifier of the medicament delivery device (10) for which the designated user triggered the medicament delivery event.
16. The receiver (11) of clause 14, further being operative to, if wireless signals are received identifying a plurality of medicament delivery devices (10, 12) communicating a medicament delivery event:
provide a recommendation to the designated user to change medicament delivery schedule.
17. The receiver (11) of any one of clauses 13-16, further being operative to, if the acquired information indicates that a wireless signal identifying said medicament delivery device (10) has been received at said at least one neighbouring receiver (13):
inform the neighbouring device (13) of the received confirmation.
18. A receiver (11) configured to associate a designated user with a medicament delivery device configured to communicate a medicament delivery event wirelessly to the receiver (11), the receiver (11) comprising a processing unit (111) and a memory (113), said memory containing instructions (112) executable by said processing unit (111), whereby the receiver (11) is operative to:
receive a wireless signal identifying a medicament delivery device (10a, 10b, 10c) communicating a medicament delivery event;
determine whether or not the identification of the medicament delivery device (10a, 10b, 10c) complies with a predetermined identification number pattern; and if so to:
determine that the identified medicament delivery device (10a, 10b, 10c) was used by the designated user for causing the medicament delivery event.
19. The receiver (11) of clause 18, further being operative to determine that the identification of the medicament delivery device (10a, 10b, 10c) complies with a predetermined identification number pattern if the identification of the medicament delivery device (10a, 10b, 10c) follows a predetermined identification number series.
20. The receiver (11) of clause 18, further being operative to determine that the identification of the medicament delivery device (10a, 10b, 10c) complies with a predetermined identification number pattern if said identification has been pre-registered with the receiver (11).

1-15. (canceled)

16. A method of a receiver of associating a designated user with a medicament delivery device configured to communicate a medicament delivery event wirelessly to the receiver, comprising:
receiving a wireless signal identifying a medicament delivery device communicating a medicament delivery event;
acquiring information indicating whether or not at least one neighbouring receiver also has received a wireless signal identifying said medicament delivery device communicating the medicament delivery event; and if not:
determining that the identified medicament delivery device was used by the designated user for causing the medicament delivery event.

17. The method of claim 16, wherein if the acquired information indicates that a wireless signal identifying said medicament delivery device has been received at said at least one neighbouring receiver, the method further comprises:
requesting a confirmation from the designated user indicating whether or not the user triggered the medicament delivery event, wherein it is determined that the identified medicament delivery device was used by the designated user for causing the medicament delivery event if a confirmation is received from the user in response to the request.

18. The method of claim 17, wherein the requesting and receiving of the confirmation is effected over a user interface of the receiver.

19. The method of claim 18, the received confirmation further comprising an identifier of the medicament delivery device for which the designated user triggered the medicament delivery event.

20. The method of claim 18, further comprising, if wireless signals are received identifying a plurality of medicament delivery devices communicating a medicament delivery event:
providing a recommendation to the designated user to change medicament delivery schedule.

21. The method of claim 16, further comprising, if the acquired information indicates that a wireless signal identifying said medicament delivery device has been received at said at least one neighbouring receiver:
informing the neighbouring device of the received confirmation.

22. A method of a receiver of associating a designated user with a medicament delivery device configured to communicate a medicament delivery event wirelessly to the receiver, comprising:
receiving a wireless signal identifying a medicament delivery device communicating a medicament delivery event;
determining whether or not the identification of the medicament delivery device complies with a predetermined identification number pattern; and if so:
determining that the identified medicament delivery device was used by the designated user for causing the medicament delivery event.

23. The method of claim 22, the identification of the medicament delivery device being determined to comply with a predetermined identification number pattern if the identification of the medicament delivery device follows a predetermined identification number series.

24. The method of claim 22, the identification of the medicament delivery device being determined to comply with a predetermined identification number pattern if said identification has been pre-registered with the receiver.

25. A computer program comprising computer-executable instructions for causing a receiver to perform steps recited in claim 16 when the computer-executable instructions are executed on a processing unit included in the receiver.

26. A computer program product comprising a computer readable medium, the computer readable medium having the computer program according to claim 25 embodied thereon.

27. A receiver configured to associate a designated user with a medicament delivery device configured to communicate a medicament delivery event wirelessly to the receiver, the receiver) comprising a processing unit and a memory, said memory containing instructions executable by said processing unit, whereby the receiver is operative to:
receive a wireless signal identifying a medicament delivery device communicating a medicament delivery event;
acquire information indicating whether or not at least one neighbouring receiver also has received a wireless signal identifying said medicament delivery device communicating the medicament delivery event; and if not to:
determine that the identified medicament delivery device was used by the designated user for causing the medicament delivery event.

28. The receiver of claim 27, further being operative to, if the acquired information indicates that a wireless signal identifying said medicament delivery device has been received at said at least one neighbouring receiver:
request a confirmation from the designated user indicating whether or not the user triggered the medicament delivery event, wherein it is determined that the identified medicament delivery device was used by the designated user for causing the medicament delivery event if a confirmation is received from the user in response to the request.

29. The receiver of claim 28, further being operative to request and receive the confirmation over a user interface of the receiver.

30. A receiver configured to associate a designated user with a medicament delivery device configured to communicate a medicament delivery event wirelessly to the receiver, the receiver comprising a processing unit and a memory, said memory containing instructions executable by said processing unit, whereby the receiver is operative to:
receive a wireless signal identifying a medicament delivery device communicating a medicament delivery event;
determine whether or not the identification of the medicament delivery device complies with a predetermined identification number pattern; and if so to:
determine that the identified medicament delivery device was used by the designated user for causing the medicament delivery event.
